(19) United States
(12) Patent Application Publication
(10) Pub. No.: US 2024/0327319 A1
(43) Pub. Date: Oct. 3, 2024

(54) RECOVERING MONO-PROPYLENE GLYCOL BY USING A DISTILLATION SOLVENT

(71) Applicant: UPM-KYMMENE CORPORATION, Helsinki (FI)

(72) Inventor: Isko KAJANTO, Espoo (FI)

(21) Appl. No.: 18/293,462

(22) PCT Filed: Sep. 5, 2022
(86) PCT No.: PCT/FI2022/050583

§ 371 Date: Jan. 30, 2024

(30) Foreign Application Priority Data
Sep. 20, 2021 (FI) 20215984

(51) Int. Cl. C07C 29/84

(52) U.S. Cl. CPC C07C 29/84

(57) ABSTRACT

A method for recovering mono-propylene glycol from a mixture feed comprising bio-derived diols and an organic impurity is disclosed. The method comprises: —providing the mixture feed into a first distillation column comprising 20-200 theoretical stages, in which first distillation column a first distillation process is carried out; —providing a distillation solvent into the first distillation column, wherein the distillation solvent is a diol or a sugar alcohol having a boiling point that is at least 80° C. higher than the boiling point of mono-propylene glycol at atmospheric pressure, and wherein the weight ratio of the distillation solvent to the total mixture feed is 2.5:1-10:1; —separating the organic impurity from mono-propylene glycol with the aid of the distillation solvent by carrying out the first distillation process at a top temperature of 70-140° C., and a top pressure of 0.01-0.2 bar, and with a reflux ratio of 2-50; and —recovering mono-propylene glycol.

### TECHNICAL FIELD

The present disclosure relates to a method for recovering mono-propylene glycol from a mixture feed comprising bio-derived diols.

### BACKGROUND

Mono-propylene glycol (MPG, also called 1,2-propanediol), is an important raw material finding use e.g. in the manufacturing of polymers. Mono-propylene glycol is a compound which is generally recognized as safe and can be further used for e.g. food applications as well as a vehicle for topical, oral and some intravenous pharmaceutical preparations. Mono-propylene glycol can be produced from propylene oxide e.g. by a non-catalytic high-temperature process at 200° C.-220° C., or by a catalytic process, which proceeds at 150° C.-180° C. in the presence of ion exchange resin or a small amount of sulfuric acid or alkali. Mono-propylene glycol can also be obtained from glycerol, a byproduct from the production of biodiesel.

In addition, mono-propylene glycol may be produced from sugars together with mono-ethylene glycol. However, when producing such polyols as mono-ethylene glycol and mono-propylene glycol from sugars also other diols, alcohols and other substances are formed as side-products. Typically, when mono-ethylene glycol is distilled from such a composition, mono-propylene glycol may be obtained as a side-product together with other lighter impurities and needs further purification. The purification of the mono-propylene glycol has however been challenging. The inventor has thus recognized the need to provide a manner for recovering purified mono-propylene glycol e.g. from the side-product when producing mono-ethylene glycol.

### SUMMARY

A method for recovering mono-propylene glycol from a mixture feed comprising bio-derived diols and an organic impurity is disclosed. The mixture feed comprises mono-propylene glycol in an amount of at least 40 weight-% of the total weight of the mixture feed. The method comprises:
providing the mixture feed into a first distillation column comprising 20-200 theoretical stages, in which first distillation column a first distillation process is carried out;
providing a distillation solvent into the first distillation column, wherein the distillation solvent is a diol or a sugar alcohol having a boiling point that is at least 80° C. higher than the boiling point of mono-propylene glycol at atmospheric pressure, and wherein the weight ratio of the distillation solvent to the total mixture feed is 2.5:1-10:1;
separating the organic impurity from mono-propylene glycol with the aid of the distillation solvent by carrying out the first distillation process at a top temperature of 70-140° C., and a top pressure of 0.01-0.2 bar, and with a reflux ratio of 2-50; and
recovering mono-propylene glycol.

### BRIEF DESCRIPTION OF THE DRAWINGS

The accompanying drawing, which is included to provide a further understanding of the embodiments and constitutes a part of this specification, illustrates an embodiment. In the drawing:

FIG. 1 discloses one embodiment of the distillation process disclosed in the current specification; and

FIG. 2 discloses one embodiment of the distillation process disclosed in the current specification.

### DETAILED DESCRIPTION

A method for recovering mono-propylene glycol from a mixture feed comprising bio-derived diols and an organic impurity is disclosed. The mixture feed comprises mono-propylene glycol in an amount of at least 40 weight-% of the total weight of the mixture feed. The method comprises:
providing the mixture feed into a first distillation column comprising 20-200 theoretical stages, in which first distillation column a first distillation process is carried out;
providing a distillation solvent into the first distillation column, wherein the distillation solvent is a diol or a sugar alcohol having a boiling point that is at least 80° C. higher than the boiling point of mono-propylene glycol at atmospheric pressure, and wherein the weight ratio of the distillation solvent to the total mixture feed is 2.5:1-10:1;
separating the organic impurity from mono-propylene glycol with the aid of the distillation solvent by carrying out the first distillation process at a top temperature of 70-140° C., and a top pressure of 0.01-0.2 bar, and with a reflux ratio of 2-50; and
recovering mono-propylene glycol.

Distillation may generally be considered a process of separating components or substances from a mixture by using selective boiling and condensation. Distillation may result in essentially complete separation into nearly pure components, or it may be a partial separation that increases the concentration of selected components in the mixture. The distillation process exploits differences in the relative volatility of the different components in the mixture.

A “theoretical stage”, a “theoretical plate” or a “distillation stage” as it may also be called, that may be used in many separation processes can be considered as a hypothetical zone or stage in which two phases, such as the liquid and vapor phases of a substance, establish an equilibrium with each other. Such equilibrium stages may also be referred to as an equilibrium stage, ideal stage, or a theoretical tray. The performance of many separation processes depends on having series of equilibrium stages and may be enhanced by providing more such stages. In other words, having more theoretical plates increases the efficiency of the separation process be it either a distillation, absorption, chromatographic, adsorption or similar process.

When designing the distillation of a certain media, the number of theoretical stages is usually first designed or considered and the theoretical stages then define the physical height of the distillation column. In the distillation column the theoretical stages or distillation stages may be formed by trays or packings, also called packed beds. A packed bed may be a structured packed bed or a random packed bed.

The inventor surprisingly found out that the combination of using the specified number of theoretical stages and the specified reflux ratio together with the distillation solvent in the specified amount enabled efficient separation of the mono-propylene from the mixture feed comprising, in addition to other bio-derived diols, the organic impurity.

comprise e.g. mono-ethylene glycol (MEG, also called ethylene glycol or 1,2-ethanediol), mono-propylene glycol (MPG, also called 1,2-propanediol), and butylene glycols (1,2-BDO, also called 1,2-butanediol and 2,3-BDO also called 2,3-butanediol) as well as an organic impurity. Such a mixture feed of bio-based diols may be derived e.g. from a process for the production of glycols, such as a process for producing mono-ethylene glycol. In one embodiment, the mixture feed comprising bio-derived diols comprises mono-ethylene glycol, mono-propylene glycol, butylene glycols, and the organic impurity. Butylene glycols may appear in structures differing from each other in where the OH-units are situated. Such structures are e.g. 1,2-butanediol and 2,3-butanediol. These have different boiling points. 1,2-butanediol has a higher boiling point and 2,3-butanediol a lower boiling point than mono-propylene glycol as presented below:

| | Boiling point (at |
|---|---|
| | atmospheric pressure) ° C. |
| 1,2-butanediol | 196.5 |
| 2,3-butanediol | 182.0 |
| mono-propylene glycol | 187.0 |
| mono-ethylene glycol | 197.1 |

The mixture feed may comprise mono-ethylene glycol, mono-propylene glycol, butylene glycols, and the organic impurity in an amount of at least 80 weight-%, or at least 85 weight-%, or at least 90 weight-%, of the total weight of the mixture feed. The mixture feed may comprise mono-propylene glycol in an amount of at least 45 weight-%, or at least 50 weight-%, or at least 55 weight-%, of the total weight of the mixture feed.

further comprise water. In one embodiment, the mixture feed comprises water in an amount of 0-8 weight-%, or 0.5-6 weight-%, or 1-5 weight-%, or 1.5-4 weight-%, or 2-3 weight, based on the total weight of the mixture feed. In one embodiment, the mixture feed comprises essentially no water.

The mixture feed may be fed into the first distillation column in the form of a liquid or as a steam or vapor, or as any mixture thereof.

Mono-ethylene glycol as well as mono-propylene glycol may be produced from a process to prepare a liquid composition of glycols comprising e.g. mono-ethylene glycol. Such a liquid composition of glycols may be prepared from plant-based raw material. The plant-based raw material may be wood-based raw material, such as from hardwood or softwood. The wood-based raw material may originate from e.g. pine, poplar, beech, aspen, spruce, eucalyptus, ash, oak, maple, chestnut, willow, or birch. The wood-based raw material may also be any combination or mixture of these.

Such a method for producing a liquid composition of glycols may comprise:
providing a wood-based feedstock originating from wood-based raw material and comprising wood chips, and subjecting the wood-based feedstock to at least one pretreatment to form a liquid fraction and a fraction comprising solid cellulose particles;
subjecting the fraction comprising solid cellulose particles to enzymatic hydrolysis to form a lignin fraction and a carbohydrate fraction;
subjecting the carbohydrate fraction to catalytical conversion to form a liquid composition of glycols.

Providing the wood-based feedstock may comprise subjecting wood-based raw material to a mechanical treatment selected from debarking, chipping, dividing, cutting, beating, grinding, crushing, splitting, screening, and/or washing the wood-based raw material to form the wood-based feedstock. Providing the wood-based feedstock may comprise purchasing the wood-based feedstock.

Pretreatment of the wood-based feedstock may comprise at least one of the following: pre-steaming of the wood-based feedstock, subjecting the wood-based feedstock to an impregnation treatment, and subjecting the wood-based feedstock to steam explosion.

The pretreatment may comprise subjecting the wood-based feedstock to pre-steaming. The pretreatment may comprise, an impregnation treatment and/or a steam explosion and may comprise, before subjecting the wood-based feedstock to impregnation treatment and/or to steam explosion, subjecting the wood-based feedstock to pre-steaming. The pre-steaming of the wood-based feedstock may be carried out with steam having a temperature of 100-130° C. at atmospheric pressure. During the pre-steaming the wood-based feedstock is treated with steam of low pressure. The pre-steaming may be also carried out with steam having a temperature of below 100° C., or below 98° C., or below 95 PC.

Further, the pretreatment may comprise subjecting the wood-based feedstock to at least one impregnation treatment with an impregnation liquid. The impregnation treatment may be carried out to the wood-based feedstock received from the mechanical treatment and/or from the pre-steaming. The pretreatment may comprise, before subjecting to the steam explosion, subjecting the wood-based feedstock to at least one impregnation treatment with an impregnation liquid selected from water, at least one acid, at least one alkali, at least one alcohol, or any combination or mixture thereof. The impregnation liquid may comprise water, at least one acid, at least one alkali, at least one alcohol, or any combination or mixture thereof.

The pretreatment may comprise subjecting the wood-based feedstock to steam explosion. The wood-based feedstock from the mechanical treatment, the pre-steaming step, and/or from the impregnation treatment may be subjected to steam explosion.

The pretreatment may comprise at least one of mechanical treatment of wood-based material to form wood-based feedstock, pre-steaming of the wood-based feedstock, impregnation treatment of the wood-based feedstock, and steam explosion of the wood-based feedstock. The pretreatment may comprise mechanical treatment of wood-based material to form a wood-based feedstock, pre-steaming of the wood-based feedstock, impregnation treatment of the pre-steamed wood-based feedstock, and steam explosion of the impregnated wood-based feedstock. The pretreatment may comprise pre-steaming the wood-based feedstock, impregnation treatment of the pre-steamed wood-based feedstock, and steam explosion of the impregnated wood-based feedstock. The pretreatment may comprise impregnation treatment of the wood-based feedstock, and steam explosion of the impregnated wood-based feedstock. I.e. the wood-based feedstock having been subjected to the impregnation treatment may thereafter be subjected to the steam explosion. Also, the wood-based feedstock having been subjected to pre-steaming, may then be subjected to the impregnation treatment and thereafter the wood-based feedstock having been subjected to the impregnation treatment may be subjected to steam explosion.

In this specification, the term “steam explosion” may refer to a process of hemihydrolysis in which the wood-based feedstock is treated in a reactor with steam having a temperature of 130-240° C. under a pressure of 0.17-3.25 MPaG followed by a sudden, explosive decompression of the steam-treated wood-based feedstock that results in the rupture of the fiber structure. The output from the steam explosion may be mixed with a suitable liquid, e.g. water, to form a slurry comprising solid cellulose particles. The fraction comprising solid cellulose particles may be separated from the liquid fraction by a suitable separation method, e.g. by a solid-liquid separation.

The enzymatic hydrolysis of the fraction comprising solid cellulose particles may be carried out at a temperature of 30-70° C., or 35-65° C., or 40-60° C., or 45-55° C., or 48-53° C. while keeping the pH of the fraction comprising solid cellulose particles at a pH value of 3.5-6.5, or 4.0-6.0, or 4.5-5.5, and wherein the enzymatic hydrolysis is allowed to continue for 20-120 h, or 30-90 h, or 40-80 h. Enzymatic hydrolysis may result in the formation of a lignin fraction and a carbohydrate fraction. The enzymes are catalysts for the enzymatic hydrolysis. The enzymatic reaction decreases the pH and by shortening the length of the cellulose fibers it may also decrease the viscosity. Subjecting the fraction comprising solid cellulose particles to enzymatic hydrolysis may result in cellulose being transformed into glucose monomers with enzymes. Lignin present in the fraction comprising solid cellulose particles may remain essentially in solid form.

At least one enzyme may be used for carrying out the enzymatic hydrolysis. The at least one enzyme may be selected from a group consisting of cellulases, hemicellulases, laccases, and lignolytic peroxidases. Cellulases are multi-protein complexes consisting of synergistic enzymes with different specific activities that can be divided into exo- and endo-cellulases (glucanase) and β-glucosidase (cellobiose). The enzymes may be either commercially available cellulase mixes or on-site manufactured.

Catalytical conversion of the carbohydrate fraction may comprise subjecting the carbohydrate fraction to catalytical hydrogenolysis. I.e. the carbohydrate fraction may be subjected to catalysts in the presence of hydrogen. The catalytical conversion may be carried out in the presence of water. In one embodiment, the catalytical conversion of carbohydrate the fraction comprises subjecting the carbohydrate fraction to catalytical hydrogenation in the presence of a solvent, preferably water and a catalyst system. The catalytical conversion may be carried out in the presence of a catalyst system comprising one or more catalysts. The catalytical conversion may alternatively be carried out to a carbohydrate feed derived from sugar cane, sugar beet, corn and/or wheat.

Subjecting the carbohydrate fraction to catalytical conversion may result in a liquid composition of glycols. The catalytical conversion accomplishes at least hydrogenolation and hydrocracking reactions to achieve hydrogenolation and hydrocracking of the carbohydrate fraction such that a liquid composition of glycols is formed. The liquid composition of glycols may comprise or consist of mono-ethylene glycol, mono-propylene glycol and butylene glycol. These glycols may be present at a concentration of 0.1-40 weight-% based on the total weight of the liquid composition of glycols. The liquid composition of glycols may also comprise other side products. The liquid composition may also comprise water.

E.g. mono-ethylene glycol may be recovered from the liquid composition of glycols e.g. by a separation technique selected form adsorption, evaporation, distillation, extractive distillation, azeotrope distillation, vacuum distillation, atmospheric distillation, membrane separation, filtration, reactive purification or a combination of them.

The mixture feed comprising bio-derived diols applied in the current specification may however also be provided from any other process for the production of glycols. The method as described in the current specification should not be understood to be bound to the above described process for producing a liquid composition of glycols.

Prior to the distillation process described in the current specification there may be one or more separation or purification processes taking place. E.g. water, alcohols such as methanol and ethanol, organic acids, sugar alcohols such as glycerol, catalysts and residual sugars may be removed in separate steps in a desired order. Typically water and alcohols having the lowest boiling point may be removed first, followed by removing components having a boiling point higher than mono-ethylene glycol. The remaining components may comprise mainly diols with boiling points close to the one of mono-propylene glycol which may then be separated in further purification steps.

By the expression “mixture feed comprising bio-derived diols” should be understood in this specification, unless otherwise stated, as a mixture feed of one or more diols, which are derived from a bio-based origin or raw material. In one embodiment, the bio-derived diols are plant-derived diols, e.g. wood-derived diols. The diols may thus be derived from e.g. hardwood, softwood, or from a combination of these. The diols may also be derived from broadleaf wood. The diols may be derived e.g. from pine, poplar, beech, aspen, spruce, eucalyptus, ash, or birch, or from any combination or mixture of these. The diols may further be derived from sugar cane, sugar beet, corn, wheat, or from any combination or mixture of these.

The inventor found out that the mixture feed comprising bio-derived diols may also comprise an organic impurity. In one embodiment, the organic impurity is characterized by a retention time of 6.5-6.7 minutes when determined by gas-chromatography-flame ionization detector (GC-FID). In one embodiment, the organic impurity is characterized by a retention time of 6.5-6.7 minutes when determined by a gas-chromatography-flame ionization detector (GC-FID) with the following parameters: The column is DB-HeavyWax (30 m×0.32 mm, 0.5 μm); the carrier gas is helium at a flow rate of 1.9 ml/min; injection temperature is 250° C. Samples are injected without dilution for identification or qualitative analysis. The starting temperature is 140° C. and the oven is kept at this temperature for 10 minutes. Then the temperature is raised to 270° C. at a heating rate of 15° C. per minute. Then the sample is kept at this temperature for 10 minutes. The total operation time is 28.67 min.

In one embodiment, the organic impurity is characterized by the tallest peak value at 59 m/z when determined by gas-chromatography-mass-spectrometer (GC-MS). In one embodiment, the organic impurity is characterized by the tallest peak value at 59 m/z when determined by gas-chromatography-mass-spectrometer (GC-MS) with the above mentioned column. The organic impurity may be further characterized by an additional peak value at 45 m/z when determined by gas-chromatography-mass-spectrometer (GC-MS).

The organic impurity may form an azeotrope with mono-propylene glycol, whereby separating it from mono-propylene glycol in order to get a high yield of pure mono-propylene glycol may be challenging. The inventor surprisingly found out that by using a specified amount of distillation solvent in the first distillation process, the azeotrope may disappear or it may be broken, such that the organic impurity and mono-propylene glycol may be at least partly separated by distillation.

An azeotrope may be considered to be a mixture that exhibits the same concentration in the vapor phase and the liquid phase. This is in contrast to ideal solutions with one component typically more volatile than the other. If the mixture forms an azeotrope, the vapor and the liquid concentrations are the same, which may prevent separation in conventional fractional distillation.

The first distillation process is carried out in a first distillation column, wherein a distillation solvent is fed to assist or aid in the separation of mono-propylene glycol from the organic impurity present in the bio-based mixture feed. The distillation solvent may further improve the separation of 1,2-butanediol and mono-ethylene glycol from mono-propylene glycol.

The distillation solvent is a diol or a sugar alcohol having a boiling point that is at least 80° C. higher than the boiling point of mono-propylene glycol at atmospheric pressure. The distillation solvent may have a boiling point that is at least 85° C., or at least 90° C., higher than the boiling point of mono-propylene glycol at atmospheric pressure. The distillation solvent may have a boiling point that is 80-100° C., or 82-98° C., or 95-95° C., higher than the boiling point of mono-propylene glycol at atmospheric pressure. The distillation solvent may have a boiling point of 265-350° C., or 265-300° C., or 275-300° C. In one embodiment, the distillation solvent is a diol having a boiling point that is at least 80° C. higher than the boiling point of mono-propylene glycol at atmospheric pressure. In one embodiment, the distillation solvent is a sugar alcohol having a boiling point that is at least 80° C. higher than the boiling point of mono-propylene glycol at atmospheric pressure.

In one embodiment, the weight ratio of the distillation solvent to the total mixture feed is 4:1-9:1, or 5:1-8:1. The inventor surprisingly found out that the specified amount of distillation solvent used in the distillation process efficiently assists in separating the organic impurity from mono-propylene glycol.

In one embodiment, the distillation solvent is tri-ethylene glycol or tri-propylene glycol. In one embodiment, the distillation solvent is tri-ethylene glycol or tri-propylene glycol. In embodiment, the one distillation solvent is tri-ethylene glycol.

The distillation solvent used has the added utility of having a boiling point higher than mono-propylene glycol and also the other diols in mixture feed. Thus, the distillation solvent used may not boil in the first distillation column and the vapor flow in the first distillation column may not increase even though a high amount of the distillation solvent is used. Thus, also the column size does not need to be increased in large extent due to the amount of the distillation solvent used.

The distillation process as disclosed in the current specification is carried out in a first distillation column. The first distillation column may comprise 20-200, or 40-120, or 40-80, or 60-120, theoretical stages. The number of theoretical stages being 20-200 has the added utility of enabling separation to take place in rather high efficiency so that reasonable reflux ratios may be used.

The mixture feed may be fed into the first distillation column at a point, which is below the point, wherein the distillation solvent is fed into the first distillation column.

The mixture feed may be fed into the first distillation column at a point, which is situated between two theoretical stages. The distillation column may comprise packings or packed beds, wherein one packed bed comprises two or more theoretical stages. In such a situation, the mixture feed may be fed into the distillation column at a point between two such packed beds. The mixture feed may be fed into the first distillation column at a point, which is situated below, above, or on at least one theoretical stage. When using plates as the theoretical stages the mixture feed may be fed on a theoretical stage or above a theoretical stage.

In one embodiment, the distillation solvent is fed into the first distillation column at any point between 1st and 10th, or 2nd and 9th, or 3rd and 7th, theoretical stages as calculated from the top of the first distillation column. The distillation solvent may be feed into the first distillation column above the topmost theoretical stage as calculated from the top of the first distillation column.

In one embodiment, the first distillation process is carried out with a reflux ratio of 3-40, or 4-30, or 5-20, or 6-10. The reflux ratio may generally be defined as the ratio of the top liquid returned to the distillation column divided by the liquid removed or recovered from the distillation column as product.

The inventor surprisingly found out that especially the combination of using the distillation solvent in the specified amount together with the other process conditions in the first distillation column has the added utility of enabling separating the mono-propylene glycol from the organic impurity present in the mixture feed and thus enabling the recovering of mono-propylene glycol with high purity and yield.

In one embodiment, method comprises recovering mono-propylene glycol with a purity of at least 80 weight-%, or at least 85 weight-%, or at least 90 weight-%, or at least 93 weight-%. In one embodiment, the mono-propylene glycol is recovered with a purity of 99.0-99.99 weight-%, or 99.3-99.95 weight-%, or 99.5-99.9 weight-%, or 99.6-99.8 weight-%. Such a purity may be achieved when a second distillation process is used after the first distillation process. The purity is calculated as the percentage of the amount of mono-propylene glycol in the recovered product compared to the total amount of recovered product flow.

In one embodiment, the yield of the mono-propylene glycol recovered is 93-100%, or 95-98%. The yield is calculated as the percentage of the amount of recovered mono-propylene glycol compared to the amount of mono-propylene glycol in the mixture feed.

In one embodiment, the first distillation process is carried out at a top temperature of 75-135° C., or 90-130° C., or 100-120 ºc.

In one embodiment, the first distillation process is carried out at a bottom temperature of 150-230° C., or 160-200° C., or 170-190° C.

In one embodiment, the first distillation process is carried out at a top pressure of 0.01-0.2 bar, or 0.015-0.1 bar, or 0.02-0.1 bar.

In one embodiment, the pressure drop over the distillation column is 0.05-0.2 bar, or 0.07-0.15 bar, or 0.08-0.1 bar.

In one embodiment, the residence time of the mixture feed and the distillation solvent in the first distillation column is 1-10 minutes, or 1.2-7 minutes, or 1.5-6 minutes, or 1.8-5.4 minutes.

The bottom temperature of the first distillation column may be kept at a temperature of at most 230° C. Keeping the bottom temperature of the distillation column at a temperature of at most 230° C. has the added utility of hindering or reducing compound degradation to take place.

In this specification, the term “top temperature” is used to refer to the temperature at the vapor space in the distillation column that is above the topmost packed bed or stage and below the vapor pipe of the distillation column. It is clear to the person skilled in the art that the temperature in the distillation column as such may differ from the temperature in e.g. the condenser or the reboiler that may be operationally connected to the distillation column. In this specification, the term “bottom temperature” is used to refer to the temperature of the liquid in the reboiler.

In this specification, the term “top pressure”, is used to refer to the pressure at the vapor space in the distillation column that is above the topmost packed bed or stage and below the vapor pipe of the distillation column.

In one embodiment, at least one condenser is used in the distillation process. In one embodiment, the distillation arrangement comprises at least one condenser. The condenser(s) used may be (a) partial condenser(s), (a) total condenser(s) or a combination of these may be used. The condenser(s) may be heat integrated or they may use a cooling medium, such as cooling water, or they may function with air cooling.

In one embodiment, a reboiler is used in the distillation process. In one embodiment, the distillation arrangement comprises a reboiler. The reboiler may be operated at a vapor pressure of 0.06-0.4 bar, or 0.1-0.2 bar.

In one embodiment, the method comprises:
removing the organic impurity together with the distillation solvent in a first bottom stream from the first distillation process; and
removing mono-propylene glycol in a first top stream from the first distillation process.

With the aid of the distillation solvent, the organic impurity will separate from mono-propylene glycol during the first distillation process and mono-propylene glycol will as a lower boiling component be distillated into the first top stream. The organic impurity separated from mono-propylene glycol, having a higher boiling point than mono-propylene glycol, may then be removed together with the distillation solvent with the first bottom stream.

In one embodiment, the method comprises recycling the distillation solvent removed in the first bottom stream from the first distillation process back into the first distillation column. From the first distillation column the distillation solvent may be led into a recovery column. In the recovery column lighter components may be removed in a second top stream from the recovery column and the distillation solvent may be removed in a second bottom stream from the recovery column. The second bottom stream comprising mainly the distillation solvent may then be led back into the first distillation column and thus reused. If needed, a part of the recycled flow of distillation solvent may be continuously purged in order to reduce or limit the accumulation of heavier degradation compounds if these appear.

In one embodiment, the method comprises providing the mono-propylene glycol removed in a first top stream from the first distillation process into a second distillation column, wherein a second distillation process is carried out. In one embodiment, the method comprises providing the mono-propylene glycol removed in a first top stream from the first distillation process into a second distillation column, wherein a second distillation process is carried out to recover mono-propylene glycol at a concentration of at least 98 weight-%, or at least 98.5 weight-%, or at least 99 weight-%.

In one embodiment, the second distillation process is carried out at a top temperature of 104-140° C., or 90-130° C., or 100-120° C.

In one embodiment, the second distillation process is carried out at a bottom temperature of 134-170° C., or 145-165° C., or 150-160° C.

In one embodiment, the second distillation process is carried out at a top pressure of 0.1-0.5 bar. In one embodiment, the second distillation process is carried out at a bottom pressure of 0.15-0.6 bar.

The method as described in the current specification has the added utility of enabling to separate the organic impurity present from mono-propylene glycol. The use of the distillation solvent in a specified amount in the first distillation process has the added utility of enabling the use of such distillation conditions that the possible azeotrope between the organic impurity and mono-propylene glycol may disappear and the separation of the organic impurity and mono-propylene glycol is possible.

### EXAMPLES

Reference will now be made in detail to various embodiments.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

For reasons of simplicity, item numbers will be maintained in the following exemplary embodiments in the case of repeating components.

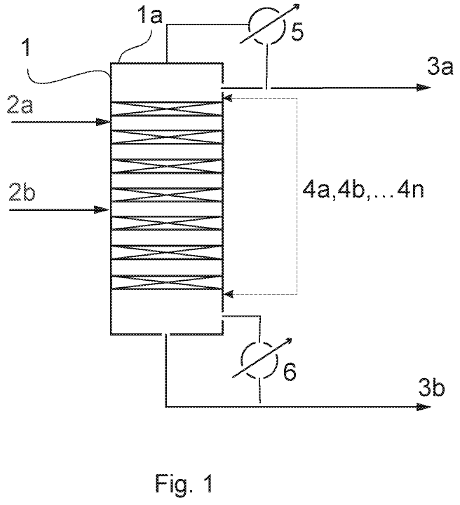

The enclosed FIG. 1 discloses an example of an embodiment for recovering mono-propylene glycol from a mixture feed comprising bio-derived diols and an organic impurity. The mixture feed 2b comprises mono-propylene glycol in an amount of at least 40 weight-% of the total weight of the mixture feed. The mixture feed is provided into a first distillation column 1, wherein a first distillation process is carried out. The first distillation column comprises an inlet 2b for feeding the mixture feed into the first distillation column 1. The first distillation column 1 comprises packed beds comprising 20-200 theoretical stages 4a, 4b, . . . 4n. The mixture feed 2b is fed into the first distillation column 1 at a point, which is lower than the point, wherein the distillation solvent 2a is fed into the first distillation column 1 as calculated from the top 1a of the distillation column. The total height of the distillation column is determined based on the number of theoretical stages 4a, 4b, . . . 4n. The first distillation column is configured to operate with a reflux ratio of 2-50, and at a top temperature of 70-140° C. and a top pressure of 0.01-0.2 bar. Mono-propylene glycol is recovered from the first distillation column 1 in a first top stream 3a. The distillation solvent and the organic impurity are removed from the first distillation column 1 in a first bottom stream 3b. FIG. 1 further presents the presence of a condenser 5 and a reboiler 6.

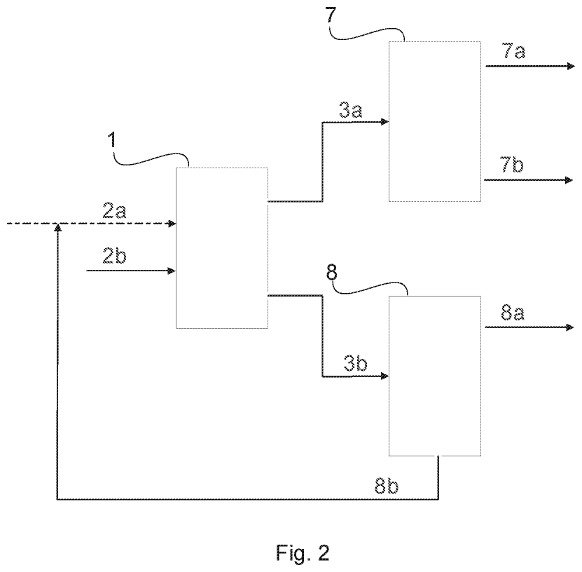

The enclosed FIG. 2 discloses an example of an embodiment for recovering mono-propylene glycol from a mixture feed comprising bio-derived diols and an organic impurity. The process begins in a similar manner as presented above in FIG. 1 with carrying out the first distillation process in the first distillation column 1. From the first distillation column 1, the mono-propylene recovered in a first top stream 3a is fed into a second distillation column 7, wherein a second distillation process is carried out. As a result of the second distillation process, mono-propylene glycol may be recovered at a concentration of at least 98 weight-% from the second bottom stream 7b. With the second top stream 7a of the second distillation column 7 is recovered 2,3-butanediol together with water and a mixture of light components. The distillation solvent removed from the first distillation column 1 in a first bottom stream 3b is fed into a recovery column 8. The distillation solvent may be purified in the recovery column 8 in order to then being recycled back in bottom recovery stream 8b to be added with feed 2a into the first distillation column 1. Any waste formed may be recovered from the recovery column 8 in a top waste stream 8a.

The distillation process will be further described in the below examples. The calculations in the below examples have been carried out with simulation made with Aspen Plus V11, using NRTL property method with Aspen database properties or estimation if values were not available. Properties for the organic impurity were generated so that there is an azeotrope with mono-propylene glycol (MPG) with the azeotropic ratio in the range that is the ratio of the organic impurity to MPG in the feed. This means an azeotropic mass ratio at 98%-99 weight-% of MPG and 1%-2 weight-% of the organic impurity. As a model compound in the simulation 2-methyl-2,3-pentanediol was used as the organic impurity as it has a very close boiling point with the MPG and forms an azeotrope with it. This way the observed non-performance of simple fractional distillation was reproduced. A RadFrac block was used for the distillation column with a total condenser.

### Example 1-Distillation of a Mixture Comprising Bio-Derived Diols

In this example, a mixture feed comprising bio-based diols and the organic impurity was subjected to the first distillation process. The following parameters were used in this example:

TABLE 1

| Number of theoretical stages | 50 | Mixture feed | |
|---|---|---|---|
| Feed stage of mixture feed | 30 | MPG | 52.82% |
| Feed stage of TEG | 5 | MEG | 20.57% |
| Reflux ratio | 5.0 | 1,2-BDO | 17.60% |
| Top pressure (bar) | 0.08 | 2,3-BDO | 3.62% |
| Column pressure drop (bar) | 0.04 | organic impurity | 0.61% |
| Mixture feed flow rate (kg/h) | 100 | water | 4.78% |
| Distillate flow (kg/h) | 58 | | |

In the above table as well as in the following examples the following meanings are abbreviations are used:
MPG=mono-propylene glycol
MEG=mono-ethylene glycol
BDO=butylene glycol
TEG=tri-ethylene glycol
   The feed stages are counted from the top of the distillation column

The results are presented below:

TABLE 2

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.52 | 64.6% | 71.0% | 114.5 | 137.9 |
| 250 | 2.5:1 | 0.47 | 84.7% | 93.0% | 113.1 | 178.9 |
| 350 | 3.5:1 | 0.42 | 84.7% | 93.1% | 113.1 | 184.1 |
| 500 | 5:1 | 0.38 | 84.8% | 93.1% | 113.1 | 188.3 |
| 750 | 7.5:1 | 0.33 | 84.9% | 93.2% | 113.1 | 191.8 |
| 1000 | 10:1 | 0.29 | 84.9% | 93.3% | 113.1 | 193.8 |

### Example 2-Distillation of a Mixture Comprising Bio-Derived Diols

In this example, a mixture feed comprising bio-based diols and the organic impurity was subjected to the first distillation process. The following parameters were used in this example:

TABLE 3

| Number of theoretical stages | 80 | Mixture feed | |
|---|---|---|---|
| Feed stage of mixture feed | 40 | MPG | 52.82% |
| Feed stage of TEG | 5 | MEG | 20.57% |
| Reflux ratio | 10.0 | 1,2-BDO | 17.60% |
| Top pressure (bar) | 0.04 | 2,3-BDO | 3.62% |
| Column pressure drop (bar) | 0.08 | organic impurity | 1.02% |
| Mixture feed flow rate (kg/h) | 100 | water | 4.37% |
| Distillate flow (kg/h) | 60 | | |

Compared to example 1, the other parameters were kept the same but the number of theoretical stages, the reflux ratio, the feed stage of the mixture feed, and the top pressure and pressure drop were varied. Also the composition of the mixture feed was different. The results are presented below:

TABLE 4

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.87 | 63.6% | 72.3% | 101.2 | 138.1 |
| 250 | 2.5:1 | 0.82 | 85.3% | 96.9% | 99.7 | 181.2 |
| 350 | 3.5:1 | 0.74 | 85.5% | 97.1% | 99.7 | 186.1 |
| 500 | 5:1 | 0.65 | 85.6% | 97.2% | 99.7 | 190.0 |
| 750 | 7.5:1 | 0.56 | 85.8% | 97.4% | 99.7 | 193.2 |
| 1000 | 10:1 | 0.49 | 85.9% | 97.5% | 99.7 | 194.9 |

### Example 3-Distillation of a Mixture Comprising Bio-Derived Diols

In this example, a mixture feed comprising bio-based diols and the organic impurity was subjected to the first distillation process. The following parameters were used in this example:

TABLE 5

| | | | |
|---|---|---|---|
| Number of theoretical stages | 100 | Mixture feed | |
| Feed stage of mixture feed | 50 | MPG | 52.82% |
| Feed stage of TEG | 5 | MEG | 20.57% |
| Reflux ratio | 10.0 | 1,2-BDO | 17.60% |
| Top pressure (bar) | 0.08 | 2,3-BDO | 3.62% |
| Column pressure drop (bar) | 0.06 | organic impurity | 1.02% |
| Mixture feed flow rate (kg/h) | 100 | water | 4.37% |
| Distillate flow (kg/h) | 60 | | |

Compared to example 2, the other parameters were kept the same but the number of theoretical stages, the feed stage of the mixture feed, and the top pressure and pressure drop were varied. The results are presented below:

TABLE 6

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.84 | 63.8% | 72.5% | 115.3 | 141.7 |
| 250 | 2.5:1 | 0.70 | 85.5% | 97.1% | 113.8 | 185.4 |
| 350 | 3.5:1 | 0.60 | 85.7% | 97.3% | 113.8 | 190.4 |
| 500 | 5:1 | 0.51 | 85.8% | 97.5% | 113.9 | 194.3 |
| 750 | 7.5:1 | 0.42 | 86.0% | 97.6% | 113.9 | 197.5 |
| 1000 | 10:1 | 0.36 | 86.0% | 97.7% | 113.9 | 199.2 |

### Example 4-Distillation of a Mixture Comprising Bio-Derived Diols

In this example, a mixture feed comprising bio-based diols and the organic impurity was subjected to the first distillation process. The following parameters were used in this example:

TABLE 7

| | | | |
|---|---|---|---|
| Number of theoretical stages | 100 | Mixture feed | |
| Feed stage of mixture feed | 50 | MPG | 52.82% |
| Feed stage of TEG | 5 | MEG | 20.57% |
| Reflux ratio | 10.0 | 1,2-BDO | 17.60% |
| Top pressure (bar) | 0.08 | 2,3-BDO | 3.62% |
| Column pressure drop (bar) | 0.06 | organic impurity | 0.61% |
| Mixture feed flow rate (kg/h) | 100 | water | 4.78% |
| Distillate flow (kg/h) | 60 | | |

Compared to example 3 the other parameters were kept the same but the composition of the mixture feed was different. The results are presented below:

TABLE 8

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.49 | 63.8% | 72.5% | 114.7 | 141.7 |
| 250 | 2.5:1 | 0.45 | 85.3% | 96.9% | 113.3 | 185.3 |
| 350 | 3.5:1 | 0.38 | 85.4% | 97.0% | 113.3 | 190.3 |
| 500 | 5:1 | 0.32 | 85.5% | 97.1% | 113.3 | 194.2 |
| 750 | 7.5:1 | 0.26 | 85.6% | 97.2% | 113.3 | 197.4 |
| 1000 | 10:1 | 0.22 | 85.6% | 97.3% | 113.3 | 199.1 |

### Example 5-Distillation of a Mixture Comprising Bio-Derived Diols

In this example, a mixture feed comprising bio-based diols and the organic impurity was subjected to the first distillation process. The following parameters were used in this example:

TABLE 9

| | | | |
|---|---|---|---|
| Number of theoretical stages | 50 | Mixture feed | |
| Feed stage of mixture feed | 30 | MPG | 52.82% |
| Feed stage of TEG | 5 | MEG | 20.57% |
| Reflux ratio | 10.0 | 1,2-BDO | 17.60% |
| Top pressure (bar) | 0.08 | 2,3-BDO | 3.62% |
| Column pressure drop (bar) | 0.04 | organic impurity | 0.61% |
| Mixture feed flow rate (kg/h) | 100 | water | 4.78% |
| Distillate flow (kg/h) | 60 | | |

Compared to example 1 the other parameters were kept the same but the reflux ratio was varied. The results are presented below:

TABLE 10

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.51 | 64.8% | 71.1% | 114.4 | 137.9 |
| 250 | 2.5:1 | 0.48 | 84.6% | 92.9% | 113.1 | 178.9 |
| 350 | 3.5:1 | 0.45 | 84.7% | 93.0% | 113.1 | 184.0 |
| 500 | 5:1 | 0.42 | 84.8% | 93.1% | 113.1 | 188.3 |
| 750 | 7.5:1 | 0.37 | 84.9% | 93.2% | 113.1 | 191.8 |
| 1000 | 10:1 | 0.34 | 84.9% | 93.2% | 113.1 | 193.8 |

### Example 6-Distillation of a Mixture Comprising Bio-Derived Diols

In this example, a mixture feed comprising bio-based diols and the organic impurity was subjected to the first distillation process. The following parameters were used in this example:

TABLE 11

| | | | |
|---|---|---|---|
| Number of theoretical stages | 100 | Mixture feed | |
| Feed stage of mixture feed | 50 | MPG | 52.82% |
| Feed stage of TEG | 5 | MEG | 20.57% |
| Reflux ratio | 5.0 | 1,2-BDO | 17.60% |
| Top pressure (bar) | 0.08 | 2,3-BDO | 3.62% |
| Column pressure drop (bar) | 0.06 | organic impurity | 1.02% |
| Mixture feed flow rate (kg/h) | 100 | water | 4.37% |
| Distillate flow (kg/h) | 60 | | |

Compared to example 3 the other parameters were kept the same but the reflux ratio was varied. The results are presented below:

TABLE 12

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.81 | 63.9% | 72.6% | 115.3 | 141.7 |
| 250 | 2.5:1 | 0.73 | 85.5% | 97.1% | 113.8 | 185.4 |
| 350 | 3.5:1 | 0.62 | 85.6% | 97.3% | 113.8 | 190.4 |
| 500 | 5:1 | 0.54 | 85.8% | 97.4% | 113.9 | 194.3 |
| 750 | 7.5:1 | 0.44 | 85.9% | 97.6% | 113.9 | 197.5 |
| 1000 | 10:1 | 0.37 | 86.1% | 97.8% | 113.9 | 199.2 |

### Example 7-Distillation of a Mixture Comprising Bio-Derived Diols

In this example, a mixture feed comprising bio-based diols and the organic impurity was subjected to the first distillation process. The following parameters were used in this example:

TABLE 13

| | | | |
|---|---|---|---|
| Number of theoretical stages | 150 | Mixture feed | |
| Feed stage of mixture feed | 75 | MPG | 52.82% |
| Feed stage of TEG | 5 | MEG | 20.57% |
| Reflux ratio | 10.0 | 1,2-BDO | 17.60% |
| Top pressure (bar) | 0.08 | 2,3-BDO | 3.62% |
| Column pressure drop (bar) | 0.06 | organic impurity | 1.02% |
| Mixture feed flow rate (kg/h) | 100 | water | 4.37% |
| Distillate flow (kg/h) | 60 | | |

Compared to example 4 the other parameters were kept the same but the number of theoretical stages, the feed stage of the mixture feed, and the pressure drop were varied. The results are presented below:

TABLE 14

| | Weight | | | | | |
|---|---|---|---|---|---|---|
| | ratio | | | | | |
| | of TEG | | | | | |
| TEG | to the | Organic | | | Top | Bottom |
| flow | total | impurity in | | | temper- | temper- |
| rate | mixture | distillate | MPG | MPG | ature | ature |
| kg/h | feed | kg/h | purity | yield | (° C.) | (° C.) |
| 0 | 0:1 | 0.73 | 58.3% | 66.3% | 115.8 | 144.3 |
| 250 | 2.5:1 | 0.49 | 85.9% | 97.5% | 113.9 | 189.2 |
| 350 | 3.5:1 | 0.40 | 86.0% | 97.7% | 113.9 | 194.2 |
| 500 | 5:1 | 0.34 | 86.1% | 97.8% | 113.9 | 198.1 |
| 750 | 7.5:1 | 0.27 | 86.2% | 97.9% | 113.9 | 201.2 |
| 1000 | 10:1 | 0.23 | 86.3% | 98.0% | 113.9 | 203.0 |

### Example 8-Distillation of a Mixture Comprising Bio-Derived Diols

In this example, a mixture feed comprising bio-based diols and the organic impurity was subjected to the first distillation process followed by the second distillation process. The mixture feed and parameters of the first distillation process were the same as in example 3 with, however, keeping the TEG flow rate at 750 kg/h.

The following parameters were used in the second distillation process:

TABLE 15

| | | | |
|---|---|---|---|
| Number of theoretical stages | 70 | Mixture of | |
| | | first top stream | |
| Feed stage of first top stream | 25 | MPG | 85.9% |
| Reflux ratio | 50 | MEG | 0.00% |
| Top pressure (bar) | 0.1 | 1,2-BDO | 0.0% |
| Column pressure drop (bar) | 0.04 | 2,3-BDO | 6.0% |
| Distillate flow (kg/h) | 8.5 | TEG | 0.0% |
| | | organic impurity | 0.7% |
| | | water | 7.3% |
| Mass flow (kg/h) | 60.00 | | |

The results are presented below:

TABLE 16

| | | MPG product | MPG distillate |
|---|---|---|---|
| | | (stream 7b | (stream 7a |
| | Units | in FIG. 2) | in FIG. 2) |
| Top temperature | ° C. | 133.0 | 49.7 |
| Top pressure | bar | 0.14 | 0.1 |
| Mass flows | kg/h | 51.50 | 8.50 |

Mass fractions

| MEG | 0.002% | 0.0 |
|---|---|---|
| MPG | 99.1% | 6.0% |
| 1,2-BDO | 0.000% | 0.0% |
| 2,3-BDO | 0.003% | 42.6% |
| TEG | 0.0% | 0.0% |
| Organic impurity | 0.9% | 0.0% |
| Water | 0.0% | 51.4% |

The mono-propylene glycol yield in this example was 97%.

### Example 9-Distillation of a Mixture Comprising Bio-Derived Diols

In this example, a mixture feed comprising bio-based diols and the organic impurity was subjected to the first distillation process followed by the second distillation process. The mixture feed and parameters of the first distillation process were the same as in example 4 with, however, keeping the TEG flow rate at 750 kg/h.

The following parameters were used in the second distillation process:

TABLE 17

| | | | |
|---|---|---|---|
| Number of theoretical stages | 70 | Mixture of | |
| | | first top stream | |
| Feed stage of first top stream | 25 | MPG | 85.6% |
| Reflux ratio | 50 | MEG | 0.00% |
| Top pressure (bar) | 0.1 | 1,2-BDO | 0.0% |
| Column pressure drop (bar) | 0.04 | 2,3-BDO | 6.0% |
| Distillate flow (kg/h) | 8.5 | TEG | 0.0% |
| | | organic impurity | 0.4% |
| | | water | 8.0% |
| Mass flow (kg/h) | 60.00 | | |

The results are presented below:

TABLE 18

| | | MPG product | MPG distillate |
|---|---|---|---|
| | | (stream 7b | (stream 7a |
| | Units | in FIG. 2) | in FIG. 2) |
| Top temperature | ° C. | 133.0 | 48.6 |
| Top pressure | bar | 0.14 | 0.1 |
| Mass flows | kg/h | 51.50 | 8.50 |

Mass fractions

| MEG | 0.003% | 0.0% |
|---|---|---|
| MPG | 99.5% | 1.3% |
| 1,2-BDO | 0.000% | 0.0% |
| 2,3-BDO | 0.028% | 42.4% |
| TEG | 0.0% | 0.0% |
| Organic impurity | 0.9% | 0.5% |
| Water | 0.0% | 56.2% |

The mono-propylene glycol yield in this example was 97%.

### Example 10-Distillation of a Mixture Comprising Bio-Derived Diols

In this example, a mixture feed comprising bio-based diols and the organic impurity was subjected to the first distillation process followed by the second distillation process. The mixture feed and parameters of the first distillation process were the same as in example 4 with, however, keeping the TEG flow rate at 750 kg/h and the distillate flow rate at 58 kg/h.

The following parameters were used in the second distillation process:

TABLE 19

| | | | |
|---|---|---|---|
| Number of theoretical stages | 70 | Mixture of | |
| | | first top stream | |
| Feed stage of first top stream | 25 | MPG | 85.2% |
| Reflux ratio | 50 | MEG | 0.00% |
| Top pressure (bar) | 0.1 | 1,2-BDO | 0.0% |
| Column pressure drop (bar) | 0.04 | 2,3-BDO | 6.2% |
| Distillate flow (kg/h) | 8.5 | TEG | 0.0% |
| | | organic impurity | 0.3% |
| | | water | 8.2% |
| Mass flow (kg/h) | 58.00 | | |

The results are presented below:

TABLE 20

| | | MPG product | MPG distillate |
|---|---|---|---|
| | | (stream 7b | (stream 7a |
| | Units | in FIG. 2) | in FIG. 2) |
| Top temperature | ° C. | 133.0 | 48.6 |
| Top pressure | bar | 0.14 | 0.1 |
| Mass flows | kg/h | 49.50 | 8.50 |

Mass fractions

| MEG | 0.003% | 0.0% |
|---|---|---|
| MPG | 99.6% | 1.3% |
| 1,2-BDO | 0.000% | 0.0% |
| 2,3-BDO | 0.03% | 42.4% |
| TEG | 0.0% | 0.0% |
| Organic impurity | 0.9% | 0.4% |
| Water | 0.0% | 56.2% |

The mono-propylene glycol yield in this example was 93%.

From the above examples and the results thereof, one can see that the larger the TEG flow rate (=weight ratio of TEG to the total mixture feed) is, the lower is the amount of the organic impurity in the distillate. Simultaneously, compared to not using any TEG in the process, the yield and the purity of the mono-propylene glycol is always higher and further the yield and purity of mono-propylene glycol are increased when the amount of TEG used in the process is increased. This trend can be seen with all numbers of theoretical stages and reflux ratios. The more theoretical stages that is used the better is the organic impurity removed.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A method disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for recovering mono-propylene glycol from a mixture feed comprising bio-derived diols and an organic impurity, wherein the mixture feed comprises mono-propylene glycol in an amount of at least 40 weight-% of the total weight of the mixture feed, and wherein the method comprises:
providing the mixture feed into a first distillation column comprising 20-200 theoretical stages, in which first distillation column a first distillation process is carried out;
providing a distillation solvent into the first distillation column, wherein the distillation solvent is a diol or a sugar alcohol having a boiling point that is at least 80° C. higher than the boiling point of mono-propylene glycol at atmospheric pressure, and wherein the weight ratio of the distillation solvent to the total mixture feed is 2.5:1-10:1;
separating the organic impurity from mono-propylene glycol with the aid of the distillation solvent by carrying out the first distillation process at a top temperature of 70-140° C. and a top pressure of 0.01-0.2 bar, and with a reflux ratio of 2-50; and
recovering mono-propylene glycol.

2. The method of claim 1, wherein the mixture feed comprises mono-propylene glycol in an amount of at least 45 weight-% of the total weight of the mixture feed.

3. The method of claim 1, wherein the mixture feed comprises mono-ethylene glycol, mono-propylene glycol, butylene glycol, and the organic impurity in an amount of at least 80 weight-% of the total weight of the mixture feed.

4. The method of claim 1, wherein the method comprises recovering mono-propylene glycol at a concentration of at least 75 weight-%.

5. The method of claim 1, wherein the weight ratio of the distillation solvent to the total mixture feed is 4:1-9:1.

6. The method of claim 1, wherein the distillation solvent is tri-ethylene glycol or tri-propylene glycol.

7. The method of claim 1, wherein the method comprises:
removing the organic impurity together with the distillation solvent in a first bottom stream from the first distillation process; and
removing mono-propylene glycol in a first top stream from the first distillation process.

8. The method of claim 1, wherein the organic impurity is characterized by a retention time of 6.5-6.7 minutes when determined by gas-chromatography-flame ionization detector (GC-FID).

9. The method of claim 1, wherein the organic impurity is characterized by the tallest peak value at 59 m/z when determined by gas-chromatography-mass-spectrometer (GC-MS).

10. The method of claim 1, wherein the first distillation column comprises 40-120 theoretical stages.

11. The method of claim 1, wherein the mixture feed is fed into the first distillation column at a point, which is below the point, wherein the distillation solvent is fed into the first distillation column.

12. The method of claim 1, wherein the first distillation process is carried out at a top temperature of 75-135° C.

13. The method of claim 1, wherein the first distillation process is carried out at a bottom temperature of 150-230° C.

14. The method of claim 1, wherein the first distillation process is carried out at a top pressure of 0.01-0.2 bar.

15. The method of claim 1, wherein the pressure drop over the distillation column is 0.05-0.2 bar, or 0.07-0.15 bar, or 0.08-0.1 bar.

16. The method of claim 1, wherein the first distillation process is carried out with a reflux ratio of 3-40, or 4-30, or 5-20, or 6-10.

17. The method of claim 1, wherein the method comprises recycling the distillation solvent removed in the first bottom stream from the first distillation process back into the first distillation column.

18. The method of claim 1, wherein the method comprises providing the mono-propylene glycol removed in a first top stream from the first distillation process into a second distillation column, wherein a second distillation process is carried out to recover mono-propylene glycol at a concentration of at least 98 weight-%.
